(19) United States
(12) Patent Application Publication
(10) Pub. No.: US 2011/0313257 A1
(43) Pub. Date: Dec. 22, 2011

(54) Method for Recording and Evaluation of Measurements Related to the Condition of a Patient for Configuration of a Portable, Patient-Controlled Device Operated to Record Said Measurements and Related Device

(76) Inventors: Klaus Abraham-Fuchs, Erlangen (DE); Maximilian Fleischer, Hohenkirchen (DE); Karsten Hiltawsky, Schwerte (DE); Oliver Hornung, Furth (DE); Thomas Krüger-Sundhaus, Pommersfelden (DE); Peter Paulicka, Rottenbach (DE); Roland Pohle, Herdweg (DE); Oliver Von Sicard, Munchen (DE)

(21) Appl. No.: 13/140,908

(22) PCT Filed: Nov. 27, 2009
(86) PCT No.: PCT/EP2009/065958

§ 371 Date: Sep. 6, 2011

(30) Foreign Application Priority Data
Dec. 19, 2008 (DE) 10 2008 064 107.3

(51) Int. Cl. A61B 5/00

(52) U.S. Cl. 600/300

(57) ABSTRACT

In a method for recording and evaluating measurements related to the condition of a patient for configuration of a portable, patient-controlled device operated to record the measurements,—at least one measurement describing the condition of the patient is recorded by the device,—the measurement and/or at least one parameter derived therefrom is/are transferred to an evaluation device via a communication connection,—the measurement and/or the parameter is/are evaluated by or on the evaluation device by an expert system and/or an expert to determine patient-specific information,—configuration data ascertained in consideration of the patient-specific information is transferred back to the device via the or another communication connection, and—the device is configured according to the configuration data.

### CROSS-REFERENCE TO RELATED APPLICATIONS

This application is a U.S. National Stage Application of International Application No. PCT/EP2009/065958 filed Nov. 27, 2009, which designates the United States of America, and claims priority to DE Application No. 10 2008 064 107.3 filed Dec. 19, 2008. The contents of which are hereby incorporated by reference in their entirety.

### TECHNICAL FIELD

The invention relates to a method for recording and evaluating measured values relating to the condition of a patient for the purpose of configuring a portable appliance which is used by the patient and which is operated for the purpose of recording the measured values, and also to an associated apparatus.

### BACKGROUND

Since it has become increasingly easy and inexpensive in recent years to produce sensors for recording measured values describing the condition of a patient, there has been an increasing number of proposals for appliances which a patient can carry in order to be able to record measured values himself. For a relatively long time, there have thus already been portable appliances used by the patient in order to make it possible for a patient himself to record his blood pressure or body temperature, for example. This allows improved management of illness which helps to avoid a large number of visits to the doctor and can also be used as a precaution. A further example of such appliances comprises an ultrasensitive nitric oxide (NO) sensor which can be used by the patient himself to make quantified recordings or even forecasts for illness progress or exacerbation, for example when asthma is present. Following appropriate consultation with a doctor, the therapy can be optimized within the correspondingly narrow time frame.

However, the problem arises in this context that, although the patient can measure the necessary measured values, such as the NO content of the breathing air as an indicator of imminent asthma attacks, himself at home, it is sometimes asking too much of him to interpret the data and the consequences which can be derived therefrom, that is to say an adjustment to the therapy or a necessary visit to the doctor, for example.

Therefore, appliances have been proposed—in addition to extensive written information to the patient—which are intended to assist the patient in interpreting the measured values. By way of example, this may involve the provision of internal criteria, particularly threshold values, which, when exceeded, can prompt the performance of an action, for example the output of a piece of information to the patient. From medical experience, such threshold values are known for measured values which might involve a potentially critical situation being present and hence it being better to pay a visit to a doctor, for example. However, the actual numerical values for threshold values may be subject to severe fluctuations interindividually and intraindividually or else may be dependent on the technical method of measurement. By way of example, one critical threshold value for an asthma attack which is being heralded is considered to be an NO value of 30 ppb in the end-expiratory breathing air, which indicates an inflammatory condition of the lung. However, it is also known that this threshold value is highly dependent on individual factors such as sex, age or body weight and that this value can also change intraindividually over the course of time as the illness progresses. The technical method of measurement which is used to gather the value, and the breathing exercise during the measurement, can also systematically influence the numerical value.

### SUMMARY

According to various embodiments, a method and an apparatus for the recording and assisted evaluation of measured values which are recorded by the patient using a portable appliance can be specified, where expert knowledge used on a patient-specific basis can be used particularly advantageously to assist the patient.

According to an embodiment, a method for recording and evaluating measured values relating to the condition of a patient for the purpose of configuring a portable appliance which is used by the patient and which is operated for the purpose of recording the measured values, may comprise the steps of: at least one measured value describing the condition of the patient is recorded using the appliance, the measured value and/or at least one variable derived therefrom is/are transmitted to an evaluation device via a communication link,—the measured value and/or the variable is/are evaluated by the or on the evaluation device in order for an expert system and/or an expert to ascertain patient-specific information,—configuration data ascertained taking account of the patient-specific information are transmitted back to the appliance via the or a further communication link, and—the appliance is configured in accordance with the configuration data, wherein an appliance which is designed to output patient information and/or warnings on the basis of at least one action criterion which evaluates the measured value, in particular on the basis of at least one threshold value, is used, wherein the appliance is provided with updated action criteria, in particular updated threshold values ascertained as part of the measured value evaluation on the evaluation device, by the configuration data.

According to a further embodiment, the appliance used can be an appliance which records a measured value relating to the lung. According to a further embodiment, the measured value measured can be an NO value, in particular end-expiratory. According to a further embodiment, the appliance used can be a handheld device or a mobile telephone, in particular with a supplementary apparatus and/or an attachment for measuring. According to a further embodiment, measured values recorded at different times can be stored and are transmitted and evaluated as a measured value profile curve. According to a further embodiment, the measured value profile curve can be evaluated as part of automatic preliminary evaluation for values characterizing the profile which are then taken into account for the evaluation on the evaluation device. According to a further embodiment, the preliminary evaluation can be performed by the appliance itself and/or by an interposed apparatus, in particular a base unit, and/or by the evaluation device. According to a further embodiment, values characterizing the profile which are ascertained can be, in particular, local maxima and/or, in particular, local minima and/or the gradient and/or a drift behavior. According to a further embodiment, the preliminary evaluation also may involve the first and/or second time derivative of the profile curve being ascertained and taken into account. According to a further embodiment, the evaluation may involve further patient-specific data, in particular the age and/ or the weight and/or the sex and/or the medical history, and/or external regional factors, in particular the weather and/or pollen counts, being taken into account. According to a further embodiment, the communication link used can be a WLAN connection and/or a Bluetooth link and/or a mobile radio link, in particular a GPRS connection. According to a further embodiment, the measured value can be accompanied by the transmission of an explicit identification number for the appliance and/or for a component of the appliance and/or for a base station explicitly associated with the appliance, in particular the MAC number of a network card in the appliance or the base station, which identification number is used in order for the evaluation device to identify the patient associated with the measured values and/or in order to ascertain the sending destination when returning. According to a further embodiment, if the measured values and/or the evaluation result satisfy at least one criterion for a possibly critical situation then the measured values and/or the evaluation result can be forwarded to a doctor. According to a further embodiment, the patient-specific information can be taken as a basis for configuring the appliance for the purpose of outputting a text message and/or for sending the patient an SMS with a text message, which text message comprises, in particular, an updated critical threshold value for the measured value. According to a further embodiment, a plurality of measured values recorded at different times can be subjected to automatic plausibility evaluation. According to a further embodiment, if nonplausibility is identified, an appliance malfunction and/or an appliance fault can be assumed and the patient and/or a third person is automatically notified thereof, particularly by means of a report on the appliance and/or an SMS and/or an e-mail.

According to another embodiment, an apparatus for the assisted self-examination of a patient, may comprise a portable appliance which is used by the patient and which is operated for the purpose of recording at least one measured value relating to the condition of a patient, which appliance is connected or can be connected to an evaluation device via a bidirectional communication link, and the evaluation device, designed to carry out the method described above.

### BRIEF DESCRIPTION OF THE DRAWINGS

Further advantages and details can be found in the exemplary embodiments described below and in the drawing, in which:

FIG. 1 shows a basic outline of an apparatus according to various embodiments, and

FIG. 2 shows a flowchart for the method according to various embodiments.

### DETAILED DESCRIPTION

According to various embodiments, a method for recording and evaluating measured values relating to the condition of a patient for the purpose of configuring a portable appliance which is used by the patient and which is operated for the purpose of recording the measured values, may comprise:
at least one measured value describing the condition of the patient is recorded using the appliance,
the measured value and/or at least one variable derived therefrom is/are transmitted to an evaluation device via a communication link,
the measured value and/or the variable is/are evaluated by the or on the evaluation device in order for an expert system and/or an expert to ascertain patient-specific information,
configuration data ascertained taking account of the patient-specific information are transmitted back to the appliance via the or a further communication link, and
the appliance is configured in accordance with the configuration data.

In this case, configuration is intended to be understood broadly within the context of various embodiments. This means that an appliance can be configured for the purpose of immediate or else delayed output of a message in precisely the same way as the alteration of internal parameters, for example of threshold values, is permanently possible. Various embodiments thus describes a method in which not only are measured values or variables derived therefrom transmitted to a central evaluation device and evaluated therein, it also proposes acknowledgement to the sending appliance in all cases, so that the method as a whole can be described as a “feedback loop”. Depending on the measured values, there is accordingly a change in the configuration of the appliance itself after bidirectional communication is advantageously used in order to further utilize the evaluation results directly for the purpose of adjusting the appliance in a way which assists the patient. This not only provides the patient with access to assistance by means of the expert knowledge, it is also possible for the expert knowledge to have a direct influence on the manner of operation of the appliance itself and in this way be used for patient-specific adaptation.

It should be noted at this juncture that the evaluation of the measured values and/or the variables derived therefrom is not intended to be understood to mean ultimate diagnosis, since such diagnosis can always be conclusively provided only by a doctor who has sufficient access to all the necessary information, in particular also to the patient himself. Hence, in some embodiments, the present method at the outside involves assisting a patient using the appliance in interpreting the measured values. It is the patient—who has detailed written instructions, for example—who ultimately takes the therapeutic consequence or can consider a visit to the doctor to be necessary. The central aspect of various embodiments is thus not determining a therapy or diagnosis on the basis of measured values but rather configuring the appliance on the basis of the feedback from an evaluation device which uses expert knowledge.

As already mentioned, the evaluation can be performed by an expert system and/or an expert, that is to say medical staff. In this case, it is ultimately preferred for the method to take place completely automatically using an expert system, the implementation of which on the appliance itself would be at least expensive and complicated, if not totally impossible.

The appliance used can be an appliance which records a measured value relating to the lung, the measured value measured advantageously being able to be an NO value, in particular end-expiratory. Particularly in the field of lung function, measured values—which may have different meanings interindividually and intraindividually—are particularly difficult to interpret. The method according to various embodiments can therefore be applied particularly advantageously to such an appliance which relates to the lung. In particular, such an appliance can be used to monitor asthma.

The appliance may be present in various forms and does not necessarily have to be used in dedicated fashion solely for recording the measured value. Thus, provision may be made for the appliance used to be a handheld device or a mobile telephone. Such standard appliances can be designed to record the measured values by virtue of supplementary apparatuses which can be connected to the appliance and which are associated with the appliance and/or by virtue of an attachment for measuring. By way of example, appliances recording measured values relating to the lung, for example, are known in which it is possible to fit a measurement attachment, into which one breathes, to the appliance. In addition, it should be noted that the portable appliance may also be provided with a base station, which is an interposed device as part of the communication. Such arrangements are widely known in the field of telephony, for example. The appliance itself then communicates, for example by means of short-range radio, with the base station, in which further functions are provided, for example pertaining to the evaluation, but particularly pertaining to the communication with the evaluation device. Such a base station or docking station can also be used to charge a battery in the appliance.

With particular advantage, provision may be made for measured values recorded at different times to be stored and to be transmitted and evaluated as a measured value profile curve. It has been found, particularly in the field of measured values relating to the lung, that the single measured value is significantly less informative for an evaluation than the profile curve for all the measured values from a plurality of measurements. By way of example, provision may be made for a patient to record a measured value, for example the NO value of the breathing air, once a day, in which case a measured value profile curve covering several weeks and months can then be considered. The individual measured values can therefore be stored, combined to form the measured value profile curve and then used for the evaluation, particularly in respect of a currently gathered new single measured value. In another form, provision may also be made for measured value profile curves to be transmitted only at particular intervals, for example after a particular number of measurements.

Expediently, the measured value profile curve can be evaluated as part of automatic preliminary evaluation for values characterizing the profile which are then taken into account for the evaluation on the evaluation device. This preliminary evaluation, which in principle corresponds to a curve discussion, does not yet require any expert knowledge, which means that the values characterizing the profile can be ascertained independently thereof. Thus, provision may be made for the preliminary evaluation to be performed by the appliance itself and/or by an interposed apparatus, particularly a base unit, and/or by the evaluation device. In the case of preliminary evaluation in a measurement appliance and/or an interposed apparatus, that is to say a base station/docking station as already discussed, for example, it is thus possible for relatively easily implemented processes to be also physically separated from the evaluation device, which is therefore under less strain, without expert knowledge. At the same time, it is conceivable for the measured values to be conditioned, ultimately by determining variables derived therefrom—since this is what the values characterizing the profile are—, such that the expert or the expert system is presented, in or on the evaluation device, precisely with the variables/values which are actually needed for the evaluation. In this way, it is also possible to reduce the number of data items which need to be transmitted via the at least one communication link. As already mentioned, the evaluation of the profile essentially corresponds to a curve discussion, which means that values characterizing the profile which are able to be ascertained are maxima, in particular local maxima, and/or minima, in particular local minima, and/or the gradient and/or drift behavior. In addition, it may be appropriate if the preliminary evaluation also involves the first and/or second time derivative of the profile curve being ascertained and taken into account. Higher derivatives are also conceivable in order to be able to assess the change in the measured value over time.

In one embodiment, the evaluation may involve further patient-specific data, in particular the age and/or the weight and/or the sex and/or the medical history, and/or external regional factors, in particular the weather and/or pollen counts, being taken into account. In the case of the already discussed cases of measured values relating to the lung, for example, there are a large number of inter-individual and also external factors which have an influence on the actual evaluation, for example when a threshold value indicating a possibly critical situation is considered in the appliance. It is therefore possible to take account of further patient-specific data, which are known in the appliance or can be obtained from a patient record, in addition to the measured values and the variables derived therefrom. In addition, it is also advantageously possible to take account of regional external factors in order to make the evaluation using the expert knowledge as exact as possible.

In order to implement the communication link, it is possible to use a WLAN connection and/or a Bluetooth link and/or a mobile radio link, particularly a GPRS connection. Of particular advantage is the use of fundamentally present and widely used communication systems, for example the Internet or mobile radio, which both provide the option of effective and fast data transmission and are also widely accessible. At this juncture, it should again be highlighted that it is also possible to use a plurality of communication links for transmitting the data to the evaluation device. By way of example, it is thus possible for a portable appliance to use radio to transmit data to a base station or docking station, which then uses a WLAN network to communicate with a further interposed apparatus, for example a PC, which in turn transmits the data to the evaluation device by means of the Internet. Arbitrary refinements are conceivable in this case.

If experts or expert knowledge is/are used in the method according to various embodiments to convince communication data, it may be appropriate for the assessment to be made on the basis of anonymized data. To allow this, provision may be made for the measured value to be accompanied by the transmission of an explicit identification number for the appliance and/or for a component of the appliance and/or for a base station explicitly associated with the appliance, in particular the MAC number of a network card in the appliance or the base station, which identification number is used in order for the evaluation device to identify the patient associated with the measured values and/or in order to ascertain the sending destination when returning. In this way, it is not necessary to transmit any kind of personal information (apart from possibly concurrently transmitted patient data such as sex, weight, etc.), particularly any name, which means that it is possible to ensure that the data are anonymous. The data are associated with the explicit identification number, which then also determines the destination to which the configuration data need to be returned.

The method according to various embodiments can also be complemented such that a doctor is immediately notified in the event of particular measured values and/or evaluation results which indicate a current critical situation which may be present. If the criterion for a possibly critical situation is met, the measured values and/or the evaluation result can be forwarded to a doctor. In the method according to various embodiments, this is done in addition to the reconfiguration of the appliance and serves to further increase patient safety.

As already set out, there are various conceivable variants for configuring the appliance by means of the configuration data. It is thus firstly conceivable for the patient-specific information to be taken as a basis for configuring the appliance for the purpose of outputting a text message and/or for sending the patient an SMS with a text message, which text message comprises, in particular, an updated critical threshold value for the measured value. In this variant, the patient receives immediate feedback relating to his measurement in the form of a text message, which may naturally also comprise further elements, but it is also conceivable, in principle, for the text message to be displayed after a delay, for example when the appliance is next used. The text message may firstly contain advice to the patient to go to a doctor to be on the safe side, or to take other measures, but it is also conceivable for the patient to be provided with evaluation assistance for the measured value or even future measured values, for example. One example of this would be the aforementioned updated critical threshold value for the measured value. An instruction to the patient regarding how to make sense of the measured values can therefore advantageously be updated on the basis of the measured values, particularly those recorded to date.

As already mentioned, it is possible to use an appliance which is designed to output patient information and/or warnings on the basis of at least one action criterion which evaluates the measured value, particularly a threshold value. If the at least one threshold value is exceeded via the measured value (or a variable derived therefrom), a piece of patient information and/or a warning is accordingly output. By way of example, it is possible to point out that a possibly critical situation is present and/or possibly imminent, so that a visit to the doctor in order to make a diagnosis may be appropriate. However, it may also be suggested that the value be reduced by taking a medicament, for example, or the patient can simply be informed about the significance of the measured value as part of an assisted evaluation. Arbitrary options are conceivable in this case.

When an appliance having such action criteria is available, provision may particularly advantageously be made for the appliance to be provided with updated action criteria, in particular updated threshold values ascertained as part of the measured value evaluation on the evaluation device, by the configuration data. In this way—possibly in addition to the configuration of the appliance for the output of the messages—the appliance is parameterized for the future, that is to say future measurements, by adjusting action criteria, particularly threshold values, in a manner specific to the patient. This embodiment can be used particularly advantageously when, as discussed further above, measured value profile curves are being considered. In this way, the action criteria are kept continually up to date by the feedback loop, and patient-specific support which is always up to date is provided for the evaluation of the measured values. If supplementary information is also taken into account, for example further patient data or external influences, the expert system allows almost all influences on the action criterion, particularly the threshold value, to be considered and to be used for continually updating the configuration of the appliance. This is particularly advantageous, again, for measured values relating to the lung, particularly for the recording of NO values, since these are subject to particular fluctuations, as noted in the introduction.

Expediently, it is additionally possible to subject a plurality of measured values recorded at different times to automatic plausibility evaluation. In this case, a check is performed to determine whether the profile of the measured values is actually plausible. The reaction provided may be that when non-plausibility is identified, an appliance malfunction and/or an appliance fault is assumed and the patient and/or a third person is automatically notified of this, particularly by means of an acknowledgement on the appliance and/or an SMS and/or an e-mail. By way of example, the patient can be asked to repeat a measurement or to initiate steps for repair or for further error analysis. By way of example, non-plausibility can be identified when values differ to a very great extent above or below the preceding value or sudden changes in the profile occur. Special criteria for non-plausibility are dependent on the nature of the recorded measured value. It goes without saying that other measures are also conceivable when non-plausibility is identified, for example the appliance can initiate a self-diagnosis or the appliance can report its possible fault or its possible malfunction to a service center by means of communication. A function check on the appliance is thus also possible when a measured value profile curve is considered.

In addition to the method, some embodiments also relate to an apparatus for the assisted self-examination of a patient, comprising a portable appliance which is used by the patient and which is operated for the purpose of recording at least one measured value relating to the condition of a patient, which appliance is connected or can be connected to an evaluation device via a bidirectional communication link, and the evaluation device, wherein the apparatus may be designed to carry out the method according to various embodiments in all of the aforementioned forms. The bidirectional communication link, which may also comprise a plurality of substeps or communication link elements, allows a feedback loop which allows the appliance to be reconfigured on the basis of the measured value and/or variables derived therefrom and/or possibly further types, that is to say to be actuated to output a text message and/or to keep action criteria up to date.

The appliance is therefore designed to transmit recorded measured values to the evaluation device, which can be done directly or else via an interposed apparatus, for example a base station, which then also forms part of the apparatus. The evaluation device is either itself designed to automatically evaluate the at least one measured value and/or the at least one variable derived therefrom, using an expert system provided on said evaluation device, in order to obtain patient-specific information or this evaluation is performed by an expert on the evaluation device. In addition, the evaluation device is designed to ascertain configuration data from the patient-specific information, which can then be transmitted back to the appliance using the bidirectional communication link. The appliance designed to receive the configuration data is then automatically reconfigured in accordance with the configuration data.

At this juncture, it should be pointed out that the bidirectional communication link can naturally be divided into a plurality of sections. Thus, by way of example, if a base station or docking station is provided for the appliance, the appliance is first able to send this base station data by radio, which data are then transmitted to the evaluation device via a further communication link, for example the Internet. The converse communication path for the bidirectional communication link then also first uses the Internet and then radio.

If a preliminary evaluation is intended to place, as described above for the method according to various embodiments, an appropriate module may be provided either as part of the appliance or as part of the evaluation device. However, such a module may also be provided on the interposed apparatus which has already been mentioned or may be intrinsically split, so that various apparatus elements can undertake different parts of the preliminary evaluation. Arbitrary refinements are conceivable in this case.

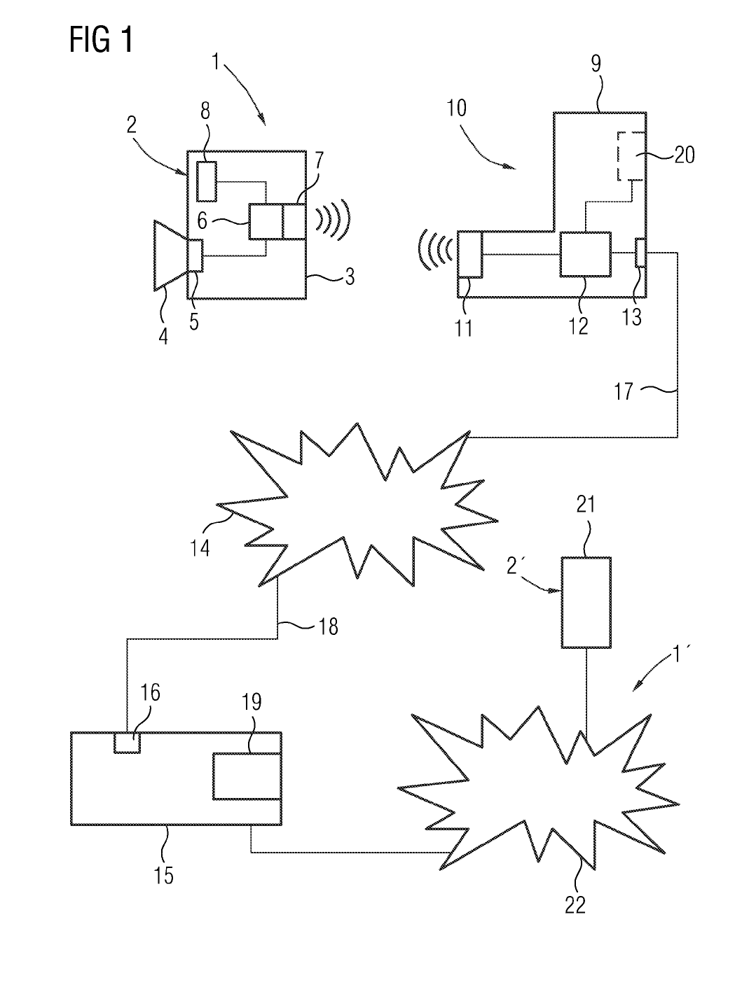

FIG. 1 shows an exemplary embodiment of an apparatus 1 for the assisted self-examination of a patient. It comprises a portable appliance 2 which is used by the patient and which, in the present case, is designed as a handheld appliance 3 with an attachment 4. The appliance 2 in the present case is used to record the NO value of the patient, that is to say a measured value relating to the lung. To this end, the appliance 2 contains an ultrasensitive NO sensor 5, wherein a patient using the appliance for measurement breathes into the attachment 4, with the NO value being measured end-expiratorily.

The appliance 2 also comprises a control device 6 having a memory—not shown in more detail in this case—which can be used to store measured values recorded at different times, a communication device 7, in this case a transmission/reception module for radio waves at a particular frequency, and a display 8.

The control device 6 also stores action criteria in the form of threshold values, with a piece of patient information and/or a warning being able to be output on the display 8 if a threshold value is exceeded by a measured NO value. In addition, a few preworded messages are provided which are output when specific configuration data are obtained, as will be discussed in more detail below. The communication device 7 is used by the appliance 2 to communicate with a base station 9, into which the appliance can also be inserted in a holder 10 for the purpose of charging a battery. The base station 9 therefore also comprises a communication device 11 which is connected to a control device 12. In addition, the base station 9 is connected to the Internet—shown symbolically at 14—by means of a connection 13. Both the control device 6 and the control device 12 may be designed to determine variables which are derived from the measured value. Measured values and/or variables derived therefrom can be forwarded via the Internet 14 to an evaluation device 15—likewise associated with the apparatus 1—with an appropriate connection 16. It should be noted that the Internet connections 17, 18 may be either WLAN connections or cabled connections. The evaluation device 16 comprises an expert system 19 which can evaluate measured values and/or variables derived therefrom for the purpose of ascertaining patient-s specific information and in so doing uses complex expert knowledge, for example by virtue of artificial intelligence. In addition, the evaluation device 15 is designed to ascertain configuration data from the patient-specific information, said configuration data being able to be transmitted to the appliance 2 or the control device 6 thereof via the Internet 14 and the communication devices 11, 7. The communication link formed by the communication devices 7 and 11 and also the Internet 14 is therefore bidirectional.

It should otherwise be pointed out that the evaluation device 15 may be designed to evaluate a measured value profile curve. It is then possible for a module to be provided which, as part of a preliminary evaluation, can ascertain values characterizing the profile, that is to say, by way of example, maxima, particularly local maxima, minima, particularly local minima, the gradient and a drift behavior, with the first, the second and possibly also higher time derivatives for the profile curve also being ascertained and taken into account. Such a module can be implemented as an electronics component or program means or else as a combination of both and provided in the appliance 2, the base station 9 or else the evaluation device 15. Such a module 20 is shown by way of example in the base station 9.

The evaluation device 15 is naturally used as a collection point for the measured values from various portable appliances 2 used by the patient, that is to say provides a central opportunity for patients to be assisted in the evaluation by expert knowledge. In this case, the appliances 2 may also be of different type and communicate with the evaluation device 15 in different ways, as indicated by the appliance 2′ shown by way of example, which is in the form of a mobile telephone 21 and which communicates with the evaluation device 15 via a mobile radio network 22. Within the context of various embodiments, the appliance 2′ and the evaluation device 15 are an apparatus 1′.

Overall, it is thus possible for an entire system comprising a plurality of appliances 2, which may be of the same type or different, to be provided which all communicate with a central evaluation device 15.

Finally, it should also be noted that it is not necessary to provide an expert system which automatically evaluates the measured values and/or the variables derived therefrom, but rather it is also possible to use an expert working on the evaluation device 15, for example medical staff.

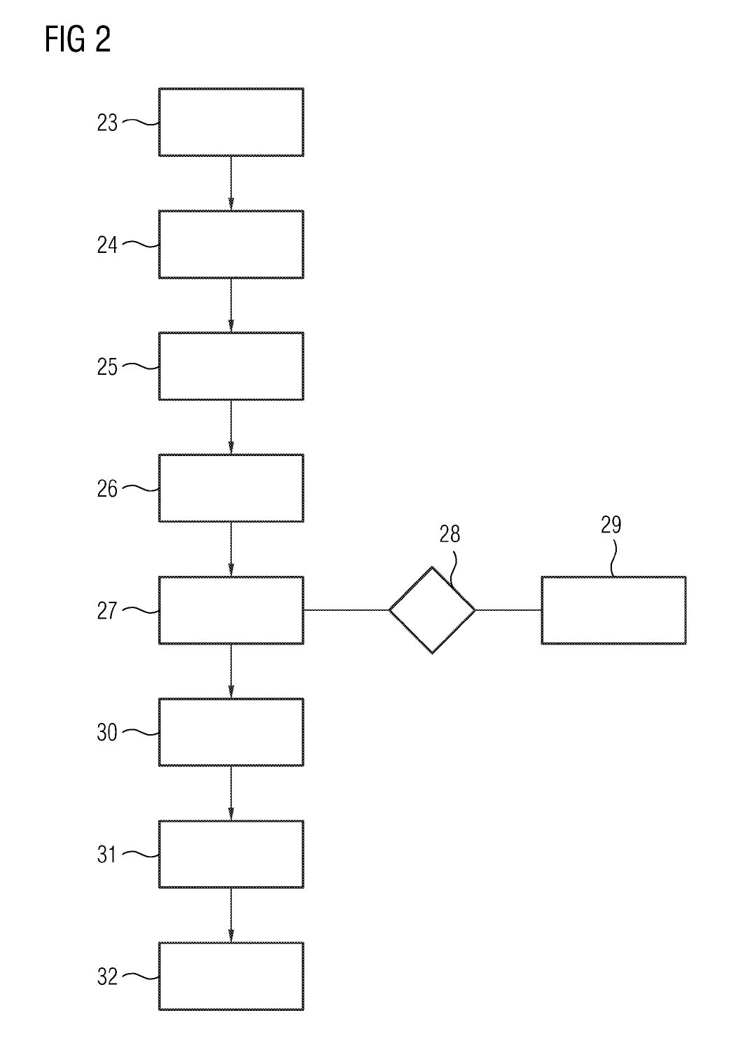

FIG. 2 shows a flowchart for an embodiment of the method which the apparatuses 1, 1′ and the system which has just been described are designed to execute.

Accordingly, a measured value, that is to say an NO value, is first recorded by the sensor 5 in a step 23. In a step 24, this measured value is stored and is therefore added to a measured value profile curve. This measured value profile curve is first of all transmitted to the base station 9 in a step 25, where preliminary evaluation takes place in the module 20. Ascertained variables derived from the measured values are now values characterizing the profile, particularly local maxima and minima, gradients, a drift behavior and also properties of the time derivatives. At this juncture, it should again be pointed out that this preliminary evaluation can also be performed in the appliance 2 itself or only in the evaluation device 15. The Internet 14 is now used to transmit the measured value profile curve and/or the variables derived therefrom to the evaluation device 15. This is done in a step 26. In a step 27, these data are evaluated in order to obtain patient-specific information. This may also involve taking account of further patient-specific data, particularly the age, weight, sex and medical history, and also external regional factors, particularly the weather and pollen counts. Whereas the regional external factors can easily be retrieved via the Internet 14, a distinction needs to be drawn in the case of the patient-specific data.

If the identity of the patient is known on the evaluation device 15, it is possible to access an electronic patient record, for example, in order to obtain the data. A different situation is present if it is important for the data to be kept anonymous and the measured values and/or the derived variables are associated by using an explicit identification number for the appliance 2, the base station 9 or a component thereof. Explicit identification of this kind can be effected using the MAC number of the network card of the base station 9, for example. It is then appropriate, if patient-specific data are intended to be used, to store said data in the appliance 2 beforehand and then to transmit them along with the measured values.

In an optional step 28, it is now possible to check whether the measured values and/or the evaluation result indicate(s) a possibly critical situation, that is to say, by way of example, whether the NO value provides indications of an inflammatory condition of the lung. It is then possible particularly in the case of values which have a particularly critical effect—for particular criteria to be taken as a basis for providing that the data are sent to a doctor, for example by means of an e-mail or an SMS, in a step 29. In a step 30, the patient-specific information is then taken into account to derive configuration data for the appliance 2. These may be either text messages which are to be output and the time at which they are output or threshold values, that is to say action criteria, which are intended to be used in future. Particularly the second variant is found to be particularly advantageous, since it is then possible for the action criteria which assist the patient in understanding the measured values to be kept continually up to date in optimized fashion when the profile of the measured values and also further patient-specific data and also external regional factors are taken into account.

These configuration data are transmitted back to the appliance 2 in a step 31, and said appliance is accordingly reconfigured in a step 32, that is to say—possibly at the particular time—outputs a text message and/or adjusts the action criteria as appropriate.

It should be pointed out that a text message may also comprise an updated threshold value which the patient can then use for evaluation when the measured values are read off from the display 8. Alternatively, the text message may indicate to the patient that it would make sense to pay a visit to the doctor, for example.

In this way, a feedback loop is produced overall which assists the patient directly or indirectly in evaluating the measured values which he has recorded. This advantageously involves the use of expert knowledge, which would not be implementable on the appliance 2.

Finally, it should also be noted that, within the context of the method according to various embodiments, it is also possible to perform an automatic plausibility evaluation by comparing measured values recorded at different, in particular successive, times. In this case, by way of example, a check is performed to determine whether a measured value appears to be too high or too low or the like overall or in comparison with the preceding or surrounding, that is to say preceding and subsequent, measured values. It is thus possible to identify appliance malfunctions, erroneous measurements and/or appliance faults. In the event of such identified non-plausibility, provision may be made for the patient and/or a third person to be automatically notified of this, particularly in an acknowledgement on the appliance, an SMS or an e-mail. In the present exemplary embodiment, this function is already implemented in the appliance 2, since this requires no expert knowledge. It is naturally equally possible for this plausibility check to be performed in the base station 9 or the evaluation device 15.

What is claimed is:

1. A method for recording and evaluating measured values relating to the condition of a patient for the purpose of configuring a portable appliance which is used by the patient and which is operated for the purpose of recording the measured values, the method comprising:
recording at least one measured value describing the condition of the patient using the appliance,
transmitting at least one of the measured value and at least one variable derived therefrom to an evaluation device via a communication link,
evaluating at least one of the measured value and the variable by the or on the evaluation device in order for at least one of an expert system and an expert to ascertain patient-specific information,
transmitting configuration data ascertained taking account of the patient-specific information back to the appliance via the or a further communication link, and
configuring the appliance in accordance with the configuration data,
wherein an appliance which is designed to output at least one of patient information and warnings on the basis of at least one action criterion which evaluates the measured value or on the basis of at least one threshold value, is used, wherein the appliance is provided with updated action criteria or updated threshold values ascertained as part of the measured value evaluation on the evaluation device, by the configuration data.

2. The method according to claim 1, wherein the appliance used is an appliance which records a measured value relating to the lung.

3. The method according to claim 2, wherein the measured value measured is an NO value, in particular end-expiratory.

4. The method according to claim 1, wherein the appliance used is a handheld device or a mobile telephone or a handheld device or mobile telephone with at least one of a supplementary apparatus and an attachment for measuring.

5. The method according to claim 1, wherein measured values recorded at different times are stored and are transmitted and evaluated as a measured value profile curve.

6. The method according to claim 5, wherein the measured value profile curve is evaluated as part of automatic preliminary evaluation for values characterizing the profile which are then taken into account for the evaluation on the evaluation device.

7. The method according to claim 6, wherein the preliminary evaluation is performed by at least one of the appliance itself by an interposed apparatus or at least one of a base unit, and by the evaluation device.

8. The method according to claim 6, wherein values characterizing the profile which are ascertained are at least one of local maxima, local minima, the gradient, and a drift behavior.

9. The method according to claim 6, wherein the preliminary evaluation also involves at least one of the first and second time derivative of the profile curve being ascertained and taken into account.

10. The method according to claim 1, wherein the evaluation involves further patient-specific data being taken into account.

11. The method according to claim 1, wherein the communication link used is at least one of a WLAN connection, a Bluetooth link, a mobile radio link, and a GPRS connection.

12. The method according to claim 1, wherein the measured value is accompanied by the transmission of an explicit identification number for at least one of the appliance, for a component of the appliance, and for a base station explicitly associated with the appliance.

13. The method according to claim 1, wherein if at least one of the measured values and the evaluation result satisfy at least one criterion for a possibly critical situation then at least one of the measured values and the evaluation result is forwarded to a doctor.

14. The method according to claim 1, wherein the patient-specific information is taken as a basis for configuring the appliance for the purpose of at least one of outputting a text message and for sending the patient an SMS with a text message, which text message.

15. The method according to claim 1, wherein a plurality of measured values recorded at different times are subjected to automatic plausibility evaluation.

16. The method according to claim 15, wherein if nonplausibility is identified, at least one of an appliance malfunction and an appliance fault is assumed and at least one of the patient and a third person is automatically notified thereof by means of at least one of a report on the appliance, and an SMS, and an e-mail.

17. An apparatus for the assisted self-examination of a patient, comprising a portable assisted self-examination of a patient, comprising a portable appliance which is used by the patient and which is operated for the purpose of recording at least one measured value relating to the condition of a patient, which appliance is connected or can be connected to an evaluation device via a bidirectional communication link, and the evaluation device, designed to
record at least one measured value describing the condition of the patient using the appliance,
transmit at least one of the measured value and at least one variable derived therefrom to an evaluation device via a communication link,
evaluate at least one of the measured value and the variable by the or on the evaluation device in order for at least one of an expert system and an expert to ascertain patient-specific information,
transmit configuration data ascertained taking account of the patient-specific information back to the appliance via the or a further communication link, and
configure the appliance in accordance with the configuration data,
wherein an appliance which is designed to output at least one of patient information and warnings on the basis of at least one action criterion which evaluates the measured value or on the basis of at least one threshold value, is used, wherein the appliance is provided with updated action criteria or updated threshold values ascertained as part of the measured value evaluation on the evaluation device, by the configuration data.

18. The method according to claim 10, wherein the further patient-specific data is at least one of the age, the weight, the sex, the medical history, external regional factors, the weather, and pollen counts.

19. The method according to claim 12, wherein the explicit identification number the MAC number of a network card in the appliance or the base station, which identification number is used in order for the evaluation device to at least one of: identify the patient associated with the measured values and in order to ascertain the sending destination when returning.

20. The method according to claim 14, wherein the text message comprises an updated critical threshold value for the measured value.
